(19) United States
(12) Patent Application Publication
(10) Pub. No.: US 2026/0315644 A1
(43) Pub. Date: Oct. 15, 2026

(54) SOLUTION FOR CONTACT LENSES

(71) Applicant: NOF Corporation, Tokyo (JP)

(72) Inventors: Hiroko KAWASAKI, Kawasaki-shi, Kanagawa (JP); Norio IWAKIRI, Kawasaki-shi, Kanagawa (JP)

(73) Assignee: NOF Corporation, Tokyo (JP)

(21) Appl. No.: 19/112,029

(22) PCT Filed: Sep. 13, 2023
(86) PCT No.: PCT/JP2023/033355

§ 371 Date: Mar. 14, 2025

(30) Foreign Application Priority Data
Sep. 15, 2022 (JP) 2022-146799

(51) Int. Cl. A61L 12/14; A61L 12/10; A61L 12/12

(52) U.S. Cl. CPC A61L 12/147; A61L 12/10; A61L 12/124

(57) ABSTRACT

To provide a solution for soft contact lens that imparts surface hydrophilicity and antifouling properties to a soft contact lens by using a solution containing a compound Q represented by the following formula (a):

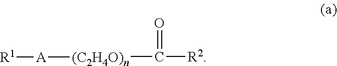

### TECHNICAL FIELD

The present invention relates to a solution for contact lens that imparts surface hydrophilicity (particularly, durable surface hydrophilicity) and antifouling properties (particularly, durable antifouling properties) to a contact lens.

### BACKGROUND ART

Due to the convenience of contact lenses for easily correcting eyesight, the number of wearers is increasing year by year, and the contact lenses have become a commonly used medical device in modern times. However, many contact lens wearers give up using the contact lenses due to the discomfort in wearing, and improving the comfort in wearing contact lenses remains a major challenge.

The discomfort in wearing the contact lenses is often described as “scratchy”, and is known to be caused by a decrease in surface wettability of the contact lenses or adhesion of dirt while the contact lenses are being worn. Further, the adhesion of dirt, particularly lipid dirt, causes the contact lenses to be cloudy, greatly reducing the eyesight. Therefore, in order to increase the comfort in wearing the contact lenses, it is important to improve the surface wettability (hydrophilicity) and antifouling properties of the contact lenses.

In order to solve the above problems, various approaches have been proposed. For example, Patent Literature 1 discloses a method of imparting surface hydrophilicity and antifouling properties to a contact lens by graft polymerization of a hydrophilic monomer into a surface of the contact lens that has been subjected to a plasma treatment. Patent Literature 2 discloses a method of imparting hydrophilicity and antifouling properties to a contact lens by treating the contact lens with a solution containing hyaluronic acid having a specific molecular weight. However, complicated steps are required in Patent Literature 1, and there is no focus on durability of effects in Patent Literature 2.

Patent Literature 3 discloses a method of cleaning lipids and preventing lipid deposition using a composition containing a nonionic polyether surfactant having an HLB of less than 12. However, this method focuses on cleaning, and does not consider lipid deposition (lipid adhesion).

### PRIOR ART DOCUMENTS

### Patent Literature

Patent Literature 1: JP2018-022174A
Patent Literature 2: WO2013/031020
Patent Literature 3: JP2007-513242T

### SUMMARY OF INVENTION

### Object to be Achieved by the Invention

An object of the present invention is to provide a solution for soft contact lens that imparts surface hydrophilicity and antifouling properties to a soft contact lens.

### Means for Achieving the Object

As a result of intensive research to solve the above problems, the inventors of the present invention have found that the above problems can be solved by using a solution containing a compound Q represented by the following formula (a). Thus, the present invention has been completed.
1. A solution for contact lens containing: a compound Q represented by the following formula (a):

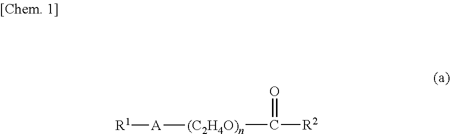

[in the formula (a), R¹ and R² each represent an alkyl group having 4 to 15 carbon atoms, A represents COO or O, and n represents 30 to 300].
2. The solution for contact lens according to the above 1, in which the solution for contact lens is a solution for shipping contact lenses.
3. The solution for contact lens according to the above 1 or 2, in which the R¹ and the R² each represent an alkyl group having 11 carbon atoms, the A represents COO, and the n represents 50 to 200.

### Effects of Invention

The solution for contact lens according to the present invention can impart durable antifouling properties and hydrophilicity to the surface of the contact lens by a simple treatment.

### EMBODIMENTS FOR CARRYING OUT THE INVENTION

### <Solution For Contact Lens>

A solution for contact lens according to the present invention is a solution for contact lens containing a compound Q represented by the following formula (a) as an effective component (main component).

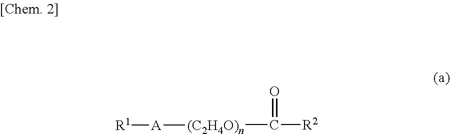

[In the formula (a), R¹ and R² each represent an alkyl group having 4 to 15 carbon atoms, and A represents COO or O. n represents 30 to 300.]

In the present description, in the case where a preferred numerical range (for example, a concentration range) is described in stages, the respective lower limit values and upper limit values can be independently combined. For example, in the description of “preferably 10 or more, more preferably 20 or more, and preferably 100 or less, more preferably 90 or less”, a “preferred lower limit value: 10” and a “more preferred upper limit value: 90” can be combined as “10 or more and 90 or less”. In addition, for example, the description of “preferably 10 to 100, more preferably 20 to 90” may also be rephrased as “10 to 90” in the same manner.

In the formula (a), n (average molar number of an oxyethylene group added) is 30 to 300, preferably 30 to 250, more preferably 50 to 200, and still more preferably 60 to 100. When n is less than 30, it is difficult to prepare a solution, and the expected effect cannot be obtained.

In addition, when n is more than 300, there is a concern that sterilization by filtration is difficult.

In the formula (a), R¹ and R² each represent an alkyl group having 4 to 15 carbon atoms, preferably an alkyl group having 5 to 15 carbon atoms, and more preferably an alkyl group having 8 to 12 carbon atoms. When the number of carbon atoms is more than 15, hydrophobicity is increased, and thereby there is a concern that the effect of improving the antifouling properties is impaired. In addition, when the number of carbon atoms is less than 3, there is a concern that adhesion to contact lenses may weaken.

In the formula (a), A is an ester (COO) or an ether (O), and is preferably an ester (COO).

Examples of the compound Q include polyethylene glycol dilaurate (R¹ and R² are each an alkyl group having 11 carbon atoms, and A is COO), polyethylene glycol dipalmitate (R¹ and R² are each an alkyl group having 15 carbon atoms, and A is COO), and polyoxyethylene myristyl ether myristate (R¹ is an alkyl group having 14 carbon atoms, R² is an alkyl group having 13 carbon atoms, and A is O). Preferred is polyethylene glycol dilaurate.

A blending amount of the compound Q in the solution for contact lens according to the present invention is not particularly limited, and is in a range of preferably 0.0001 w/v % to 0.3 w/v %, more preferably 0.001 w/v % to 0.2 w/v % and still more preferably 0.01 w/v % to 0.1 w/v %.

When the content is less than 0.0001 w/v %, there is a possibility that a sufficient effect cannot be obtained, and when the content is more than 0.3 w/v %, there is a possibility that the solution for contact lens is difficult to handle during production due to an increase in viscosity and generation of foaming.

### <Additive>

The solution for contact lens according to the present invention may contain, in addition to the compound Q which is an essential component (effective component), an additive and the like which are used in the preparation of ordinary solutions for contact lens, if necessary.

A blending amount of the additive is not particularly limited, and is preferably 0.01 w/v % to 5.0 w/v %, more preferably 0.05 w/v % to 4.0 w/v %, and still more preferably 0.1 w/v % to 3.0 w/v %.

Examples of the additive include vitamins, a thickener, amino acids, sugars, a cooling agent, inorganic salts, organic acid salts, an antioxidant, a stabilizer, a preservative, a chelating agent, and a buffering agent.

Examples of the vitamins include flavin adenine dinucleotide sodium, cyanocobalamin, retinol acetate, retinol palmitate, pyridoxine hydrochloride, panthenol, sodium pantothenate, and calcium pantothenate.

Examples of the thickener include hydroxypropyl methylcellulose, polyvinyl alcohol, sodium hyaluronate, alginic acid, guar gum, cellulose, and a derivative thereof.

Examples of the amino acids include aspartic acid and salts thereof, and aminoethylsulfonic acid.

Examples of the sugars include glucose, mannitol, sorbitol, xylitol, and trehalose.

Examples of the cooling agent include menthol and camphor.

Examples of the inorganic salts include sodium chloride and potassium chloride.

Examples of the organic acid salts include sodium citrate.

Examples of the antioxidant include tocopherol acetate, taurine, sodium bisulfite, sodium sulfite, and dibutylhydroxytoluene.

Examples of the stabilizer include sodium decanoate and glycine.

Examples of the preservative include benzalkonium chloride, chlorhexidine gluconate, potassium sorbate, polyhexanide hydrochloride, paraben, and chlorobutanol.

Examples of the chelating agent include EDTA-2Na, trihydroxymethylaminomethane, and sodium hexametaphosphate.

Examples of the buffering agent include a borate buffer solution, a phosphate buffer solution, and a citrate buffer solution. Further, the phosphate buffer solution is a buffer solution containing a component selected from disodium hydrogen phosphate, sodium dihydrogen phosphate, potassium dihydrogen phosphate, dipotassium hydrogen phosphate, and a hydrate thereof, and hydrochloric acid, sodium hydroxide, and potassium hydroxide. The borate buffer solution is a buffer solution containing a component selected from boric acid, borax, hydrochloric acid, sodium hydroxide, and potassium hydroxide.

### <Method for Producing Solution For Contact Lens>

The solution for contact lens according to the present invention can be produced by using a general method for producing a solution for contact lens, in which the compound Q, which is an essential component, water, and other components (additives), if necessary, are mixed and stirred. During the production, a heating and stirring step may be added depending on the solubility of the other components added. Note that, the obtained solution for contact lens may be subjected to an operation such as sterilization filtration, if necessary.

### <pH of Solution for Contact Lens>

The solution for contact lens according to the present invention has a pH of preferably 3 to 9, more preferably 3 to 8, and still more preferably 4 to 8, from the viewpoint of improving the comfort in wearing contact lenses.

Note that, the pH of the solution for contact lens in the present description refers to a value measured in accordance with 2.54 pH Determination of General Tests in the Japanese Pharmacopoeia 18th Edition.

### <Osmolarity and Osmolarity Ratio of Solution For Contact Lens>

The solution for contact lens according to the present invention has an osmolarity of preferably 200 mOsm to 400 mOsm, more preferably 210 mOsm to 350 mOsm, and still more preferably 220 mOsm to 340 mOsm, and has an osmolarity ratio of preferably 0.7 to 1.4, more preferably 0.8 to 1.3, and still more preferably 0.8 to 1.2, from the viewpoint of improving the comfort in wearing contact lenses.

Note that, the osmolarity of the solution for contact lens in the present description refers to the value measured in accordance with 2.47 Osmolarity Determination (Osmolar Concentration Determination) of General Tests in the Japanese Pharmacopoeia 18th Edition, and the osmolarity ratio refers to the value obtained by dividing the obtained osmolarity value by the osmolarity value (286 mOsm) of 0.9 mass % physiological saline.

### <Use Form of Solution For Contact Lens>

Specific examples of a use form of the solution for contact lens according to the present invention include a solution for shipping contact lenses, a contact lens care product, a disinfectant for contact lenses, a solution for storing contact lenses, a solution for cleaning contact lenses, a solution for cleaning and storing contact lenses, a solution for sterilizing, cleaning, and storing contact lenses, and a contact lens wearing solution.

When the solution is used as a solution for shipping contact lenses, contact lenses can be immersed in the solution for contact lens according to the present invention, sealed, and then heat sterilized.

Hereinafter, the present invention will be described in more detail with reference to Examples and Comparative Examples, but the present invention is not limited thereto.

### EXAMPLE 1-1

About 80 g of purified water was weighed out, and 0.005 g of polyethylene glycol dilaurate (average molar number of an oxyethylene group added: 70), 0.83 g of sodium chloride, 0.053 g of sodium dihydrogen phosphate dihydrate, and 0.599 g of disodium hydrogen phosphate dodecahydrate were weighed out and dissolved therein. Thereafter, purified water was added to make the total volume 100 mL. The solution was sterilized by filtration to obtain a sterile solution for contact lens according to the present invention (particularly, a solution for shipping contact lenses). The solution for contact lens thus obtained was evaluated for the effect of improving the hydrophilicity of the contact lens and the effect of improving the antifouling properties of the contact lens, to be described later. The results are shown in Table 1.

### EXAMPLE 1-2 TO EXAMPLE 1-4 AND COMPARATIVE EXAMPLE 1-1 TO COMPARATIVE EXAMPLE 1-4

Sterile solutions for contact lens (particularly, solution for shipping contact lenses) were each prepared in the same manner as in Example 1, except that the types and the amounts of components shown in Table 1 or Table 2 were used. Each of the solutions for contact lens thus obtained was evaluated for the effect of improving the hydrophilicity of the contact lens and the effect of improving the antifouling properties of the contact lens, to be described later. The results are shown in Table 1 and Table 2.

### EXAMPLE 2-1 TO EXAMPLE 2-4 AND COMPARATIVE EXAMPLE 2-1 TO COMPARATIVE EXAMPLE 2-4

According to the formulation examples shown in Table 3 and Table 4, solutions for contact lens according to the present invention (particularly, contact lens care products) were prepared. The contact lens care products were prepared in the same manner as in Example 1-1, except that the types and the amounts shown in Table 3 and Table 4 were used. Each of the contact lens care products were evaluated for the effect of improving the hydrophilicity of the contact lens and the effect of improving the antifouling properties of the contact lens, to be described later. The results are shown in Table 3 and Table 4.

### <Examination on Effect of Improving Hydrophilicity of Contact Lens>

In Examples and Comparative Examples, the hydrophilicity of the contact lenses was evaluated according to the following procedure. Note that, in order to examine the effect of improving the hydrophilicity of the contact lens, Polymacon (a non-ionic low water content material) lenses were used.

### (Procedure)

1) The solution for contact lens in Examples or Comparative Examples was used.
2) One contact lens was charged into a 15 mL centrifuge tube.
3) 10 mL of physiological saline solution was added to the centrifuge tube in 2) and shaken overnight.
4) The physiological saline solution was removed from the centrifuge tube in 3), and 10 mL of the solution for contact lens in 1) was added to the centrifuge tube.
5) The centrifuge tube in 4) was autoclaved under conditions of 121° C. for 20 minutes. However, this operation was not carried out in Example 2-1 to Example 2-4 and Comparative Example 2-1 to Comparative Example 2-4.
6) After cooling to room temperature, the contact lens was taken out and a time (BUT) until the water film on the lens surface disappeared was measured with a stopwatch. Those having a BUT of 10 seconds or longer were evaluated as having “excellent surface hydrophilicity” and were marked with “B” in Table 1, and those having a BUT of 15 seconds or longer were evaluated as having “particularly excellent surface hydrophilicity” and were marked with “A” and those having a BUT of shorter than 10 seconds were marked with “C” in Table 1 to Table 4.

### <Evaluation of Durability of Effect of Improving Hydrophilicity of Contact Lens>

### (Procedure)

1) The solution for contact lens in Examples or Comparative Examples was used.
2) One contact lens was charged into a 15 mL centrifuge tube.
3) 10 mL of physiological saline solution was added to the centrifuge tube in 2) and shaken overnight.
4) The physiological saline solution was removed from the centrifuge tube in 3), and 10 mL of the solution for contact lens in 1) was added to the centrifuge tube.
5) The centrifuge tube in 4) was autoclaved under conditions of 121° C. for 20 minutes. However, this operation was not carried out in Example 2-1 to Example 2-4 and Comparative Example 2-1 to Comparative Example 2-4.
6) After cooling to room temperature, the contact lens was taken out and the contact lens was then immersed in 2 mL of physiological saline solution and washed with shaking for 30 minutes.
7) The remaining operations were carried out and evaluation was carried out in the same manner as in <Examination on Effect of Improving Hydrophilicity of Contact Lenses>. The evaluation results are shown in Table 1 to Table 4.

### <Evaluation of Effect of Improving Antifouling Properties of Contact Lens>

In Examples and Comparative Examples, the antifouling properties of the contact lenses were evaluated according to the following procedure. Note that, in order to examine the effect of improving the antifouling properties of the contact lens, ACUVUEOASYS (registered trademark) (manufactured by Johnson & Johnson) was used.

### (Procedure)

### Preparation of Artificial Rheum

1) 0.5 g of a lipid mixture having the composition shown below was mixed with 100 mL of a phosphate-borate buffer solution shown below.
2) The mixture was suspended at 60° C. using a homomixer.
3) The pH was adjusted to 7.0 with 1N hydrochloric acid.
   Composition of lipid mixture
Oleic acid: 0.06 g
Linolenic acid: 0.06 g
Palmitic acid: 0.06 g
Tripalmitic acid: 0.81 g
Cetyl alcohol: 0.20 g
Cetyl myristate: 0.81 g
Cholesterol: 0.08 g
Cholesterol palmitate: 0.08 g
Lecithin (derived from egg): 2.83 g
Composition of phosphate-borate buffer solution
Sodium chloride: 2.25 g
Potassium dihydrogen phosphate: 1.25 g
Sodium tetraborate decahydrate: 5.65 g
Ion exchange water: 250 mL in total amount

### (Evaluation)

1) The solution for contact lens in Examples or Comparative Examples was used.
2) One contact lens was charged into a 15 mL centrifuge tube.
3) 10 mL of physiological saline solution was added to the centrifuge tube in 2) and shaken overnight.
4) Thereafter, the physiological saline solution was removed, and 10 mL of the solution for contact lens in 1) was added to the centrifuge tube in 3).
5) An autoclave treatment was carried out under conditions of 121° C. for 20 minutes. However, this operation was not carried out in Example 2-1 to Example 2-4 and Comparative Example 2-1 to Comparative Example 2-4.
6) After the contact lens was cooled to room temperature, one contact lens was immersed in 4 mL of artificial rheum for 4 hours.
7) The contact lens was removed, lightly rinsed with a physiological saline solution, and dried.
8) The contact lens was immersed in 3 mL of an extraction solution (ethanol/diethyl ether=1/1 (v/v)).
9) An ultrasonic treatment was carried out for 10 minutes.
10) The amount of lipids in the extraction solution was quantified by using the vanillin-phosphoric acid method (see Ii Kaoru, Moriyama Mari, Imayasu Masaki, Tanaka Hidenari: Study on lipid adhesion to contact lenses, Journal of Japan Contact Lens Society, vol. 37, pp. 58-61, 1995).

The amount of lipids adhering to the contact lens was determined by comparing the absorbance obtained with a calibration curve. A similar test was carried out using a physiological saline solution as the solution for contact lens, and a lipid adhesion inhibition rate was calculated based on the amount of lipid adhesion measured. A case where the lipid adhesion inhibition rate was 25% or more was evaluated as “having adhesion inhibition ability”, and a case where the lipid adhesion inhibition rate was 50% or more was evaluated as “having particularly excellent adhesion inhibition ability”. The evaluation results are shown in Table 1 to Table 4.

### <Evaluation of Durability of Effect of Improving Antifouling Properties of Contact Lens>

### (Procedure)

1) The solution for contact lens in Examples or Comparative Examples was used.
2) One contact lens was charged into a 15 mL centrifuge tube.
3) 10 mL of physiological saline solution was added to the centrifuge tube in 2) and shaken overnight.
4) Thereafter, the physiological saline solution was removed, and 10 mL of the solution for contact lens in 1) was added to the centrifuge tube in 3).
5) An autoclave treatment was carried out under conditions of 121° C. for 20 minutes. However, this operation was not carried out in Example 2-1 to Example 2-4 and Comparative Example 2-1 to Comparative Example 2-4.
6) After cooling the contact lens to room temperature, the contact lens was taken out and the contact lens was then immersed in 2 mL of physiological saline solution and washed with shaking for 30 minutes.
7) One contact lens was immersed in 4 mL of artificial rheum for 4 hours.
8) The contact lens was removed, lightly rinsed with a physiological saline solution, and dried.
9) The contact lens was immersed in 3 mL of an extraction solution (ethanol/diethyl ether=1/1 (v/v)).
10) An ultrasonic treatment was carried out for 10 minutes.
11) The amount of lipids in the extraction solution was quantified by using the vanillin-phosphoric acid method.

Based on the obtained absorbance, the lipid adhesion inhibition rate was calculated in the same manner as in <Evaluation of Effect of Improving Antifouling Properties of Contact Lens>. The evaluation results are shown in Table 1 to Table 4.

TABLE 1

| | | Example 1-1 | Example 1-2 | Example 1-3 | Example 1-4 |
|---|---|---|---|---|---|
| Essential | Polyethylene glycol | 0.005 | 0.001 | | |
| component | dilaurate (average | | | | |
| | EO: 70) | | | | |
| | Polyethylene glycol | | | 0.005 | 0.001 |
| | dilaurate (average | | | | |
| | EO: 150) | | | | |
| Blending | Sodium chloride | 0.83 | 0.4 | 0.83 | 0.2 |
| amount | Potassium chloride | | 0.35 | | 0.6 |
| | Boric acid | | 0.4 | | 0.4 |
| | Borax | | 0.015 | | |
| | Sodium hydroxide | | | | 0.0072 |
| | Disodium hydrogen | 0.599 | | 0.599 | |
| | phosphate | | | | |
| | dodecahydrate | | | | |
| | Sodium dihydrogen | 0.053 | | 0.053 | |
| | phosphate dihydrate | | | | |
| | Purified water | 100 mL in | 100 mL in | 100 mL in | 100 mL in |
| | | total amount | total amount | total amount | total amount |
| Analysis | Property and | Colorless | Colorless | Colorless | Colorless |
| result | appearance | and clear | and clear | and clear | and clear |
| | pH | 7.2 | 7.0 | 7.2 | 7.2 |
| | Osmolarity | 310 | 296 | 311 | 295 |
| Evaluation | Evaluation of | A | A | A | A |
| result | hydrophilicity | | | | |
| | Evaluation of | A | B | B | B |
| | hydrophilicity | | | | |
| | (durability) | | | | |
| | Evaluation of | 89 | 50 | 85 | 47 |
| | antifouling | | | | |
| | properties | | | | |
| | Evaluation of | 71 | 37 | 57 | 40 |
| | antifouling | | | | |
| | properties | | | | |
| | (durability) | | | | |

TABLE 2

| | | Comparative | Comparative | Comparative | Comparative |
|---|---|---|---|---|---|
| | | Example 1-1 | Example 1-2 | Example 1-3 | Example 1-4 |
| Comparative | Polyethylene glycol | 0.1 | | | 0.05 |
| component | monostearate (average | | | | |
| | EO: 75) | | | | |
| | Polyethylene glycol | | 0.1 | | |
| | dilaurate (average | | | | |
| | EO: 9) | | | | |
| | Polyethylene glycol | | | 0.1 | |
| | 200 | | | | |
| Blending | Sodium chloride | 0.83 | 0.4 | 0.83 | 0.2 |
| amount | Potassium chloride | | 0.35 | | 0.6 |
| | Boric acid | | 0.4 | | 0.4 |
| | Borax | | 0.015 | | |
| | Sodium hydroxide | | | | 0.0072 |
| | Disodium hydrogen | 0.599 | | 0.599 | |
| | phosphate dodecahydrate | | | | |
| | Sodium dihydrogen | 0.053 | | 0.053 | |
| | phosphate dihydrate | | | | |
| | Purified water | 100 mL in | 100 mL in | 100 mL in | 100 mL in |
| | | total amount | total amount | total amount | total amount |
| Analysis | Property and | Colorless | Insoluble | Colorless | Colorless |
| result | appearance | and clear | | and clear | and clear |
| | pH | 7.2 | — | 7.2 | 7.2 |
| | Osmolarity | 310 | — | 310 | 282 |
| Evaluation | Evaluation of | B | — | B | B |
| result | hydrophilicity | | | | |
| | Evaluation of | B | — | C | C |
| | hydrophilicity | | | | |
| | (durability) | | | | |
| | Evaluation of | −69 | — | 90 | −32 |
| | antifouling | | | | |
| | properties | | | | |
| | Evaluation of | −40 | — | 97 | −25 |
| | antifouling | | | | |
| | properties | | | | |
| | (durability) | | | | |

*In the evaluation of antifouling properties, − (minus) indicates that the amount of adhesion is greater than that of the physiological saline solution.

TABLE 3

| | | Example 2-1 | Example 2-2 | Example 2-3 | Example 2-4 |
|---|---|---|---|---|---|
| Essential | Polyethylene glycol | 0.1 | 0.005 | | |
| component | dilaurate (average | | | | |
| | EO: 70) | | | | |
| | Polyethylene glycol | | | 0.1 | 0.005 |
| | dilaurate (average | | | | |
| | EO: 150) | | | | |
| Blending | Sodium chloride | 0.5 | 0.55 | 0.5 | 0.55 |
| amount | Potassium chloride | 0.1 | | 0.1 | |
| | Boric acid | 0.4 | 0.4 | 0.4 | 0.4 |
| | Borax | | | | |
| | Sodium hydroxide | 0.003 | 0.007 | 0.003 | 0.007 |
| | Disodium hydrogen | | | | |
| | phosphate | | | | |
| | dodecahydrate | | | | |
| | Sodium dihydrogen | | | | |
| | phosphate dihydrate | | | | |
| | Hydroxypropyl | 0.05 | 0.05 | 0.05 | 0.05 |
| | methylcellulose | | | | |
| | Sodium hyaluronate | 0.05 | 0.05 | 0.05 | 0.05 |
| | 20% Polyhexanide | 0.05 | 0.05 | 0.05 | 0.05 |
| | hydrochloride | | | | |
| | aqueous solution | | | | |
| | Purified water | 100 mL in | 100 mL in | 100 mL in | 100 mL in |
| | | total amount | total amount | total amount | total amount |
| Analysis | Property and | Colorless | Colorless | Colorless | Colorless |
| result | appearance | and clear | and clear | and clear | and clear |
| | pH | 6.8 | 7.2 | 7.2 | 7.2 |
| | Osmolarity | 263 | 253 | 311 | 290 |
| Evaluation | Evaluation of | A | A | A | A |
| result | hydrophilicity | | | | |
| | Evaluation of | A | B | A | B |
| | hydrophilicity | | | | |
| | (durability) | | | | |
| | Evaluation of | 91 | 85 | 92 | 82 |
| | antifouling | | | | |
| | properties | | | | |
| | Evaluation of | 60 | 55 | 63 | 56 |
| | antifouling | | | | |
| | properties | | | | |
| | (durability) | | | | |

EO: average molar number of oxyethylene moiety added

TABLE 4

| | | Comparative | Comparative | Comparative | Comparative |
|---|---|---|---|---|---|
| | | Example 2-1 | Example 2-2 | Example 2-3 | Example 2-4 |
| Comparative | Polyethylene glycol | 0.1 | | | |
| component | monostearate (average | | | | |
| | EO: 70) | | | | |
| | Polyethylene glycol | | 0.1 | | |
| | dilaurate (average | | | | |
| | EO: 9) | | | | |
| | Polyethylene glycol | | | 0.1 | 0.005 |
| | 200 | | | | |
| Blending | Sodium chloride | 0.5 | 0.5 | 0.5 | 0.55 |
| amount | Potassium chloride | 0.1 | 0.1 | 0.1 | |
| | Boric acid | 0.4 | 0.4 | 0.4 | 0.4 |
| | Borax | | | | |
| | Sodium hydroxide | 0.003 | 0.003 | 0.003 | 0.007 |
| | Disodium hydrogen | | | | |
| | phosphate | | | | |
| | dodecahydrate | | | | |
| | Sodium dihydrogen | | | | |
| | phosphate dihydrate | | | | |
| | Hydroxypropyl | 0.05 | 0.05 | 0.05 | 0.05 |
| | methylcellulose | | | | |
| | Sodium hyaluronate | 0.05 | 0.05 | 0.05 | 0.05 |
| | 20% Polyhexanide | 0.05 | 0.05 | 0.05 | 0.05 |
| | hydrochloride | | | | |
| | aqueous solution | | | | |
| | Purified water | 100 mL in | 100 mL in | 100 mL in | 100 mL in |
| | | total amount | total amount | total amount | total amount |
| Analysis | Property and | Colorless | Insoluble | Colorless | Colorless |
| result | appearance | and clear | | and clear | and clear |
| | pH | 6.8 | — | 6.8 | 7.2 |
| | Osmolarity | 263 | — | 263 | 290 |
| Evaluation | Evaluation of | B | — | B | C |
| result | hydrophilicity | | | | |
| | Evaluation of | C | — | C | C |
| | hydrophilicity | | | | |
| | (durability) | | | | |
| | Evaluation of | −40 | — | 8 | 9 |
| | antifouling | | | | |
| | properties | | | | |
| | Evaluation of | −24 | — | 4 | −1 |
| | antifouling | | | | |
| | properties | | | | |
| | (durability) | | | | |

*In the evaluation of antifouling properties, − (minus) indicates that the amount of adhesion is greater than that of the physiological saline solution.

### Evaluation Results

### Examples 1-1 to 1-4

The surface hydrophilicity of the contact lens turns out to be particularly excellent. Further, the durable surface hydrophilicity turns out to be particularly excellent or excellent.

The antifouling properties of the contact lens turn out to be particularly excellent or excellent in terms of adhesion inhibition ability. Further, the durable antifouling properties of the contact lens similarly turn out to be particularly excellent or excellent in terms of adhesion inhibition ability.

As seen from the results in Example 1-1 and Example 1-3, polyethylene glycol dilaurate (EO: about 70), which is an essential component of the solution for contact lens according to the present invention, is more excellent than polyethylene glycol dilaurate (EO: about 150).

It can be seen that the solution for contact lens according to the present invention also has an excellent effect as a solution for shipping contact lenses.

### Examples 2-1 to 2-4

The surface hydrophilicity of the contact lens turns out to be particularly excellent. Further, the durable surface hydrophilicity turns out to be particularly excellent or excellent.

The antifouling properties of the contact lens turn out to be particularly excellent in terms of adhesion inhibition ability. Further, the durable antifouling properties of the contact lens similarly turn out to be particularly excellent in terms of adhesion inhibition ability.

It can be seen that the solution for contact lens according to the present invention also has an excellent effect as a contact lens care product.

### Comparative Examples 1-1 to 1-4

Since Comparative Example 1-2 is insoluble, no evaluation was carried out.

Regarding Comparative Examples 1-1, 1-3, and 1-4, no effect can be obtained in any one of the surface hydrophilicity, the durable surface hydrophilicity, the antifouling properties, and the durable antifouling properties.

### Comparative Examples 2-1 to 2-4

Since Comparative Example 2-2 is insoluble, no evaluation was carried out.

It can be seen that Comparative Examples 2-1, 2-3, and 2-4 have no durable surface hydrophilicity, antifouling properties, or durable antifouling properties.

The solution for contact lens according to the present invention can impart durable antifouling properties and hydrophilicity to the surface of the contact lens by a simple treatment, and can impart good comfort and eyesight when the contact lens is worn.

### INDUSTRIAL APPLICABILITY

It is possible to provide a solution for contact lens that imparts durable surface hydrophilicity and antifouling properties to a contact lens.

Although the present invention has been described in detail and with reference to specific embodiments, it will be apparent to those skilled in the art that various changes and modifications can be made without departing from the spirit and scope of the present invention.

The present application is based on a Japanese patent application (Japanese Patent Application No. 2022-146799) filed on Sep. 15, 2022, the contents of which are incorporated herein by reference.

1. A solution for contact lens comprising:
a compound represented by the following formula (a): 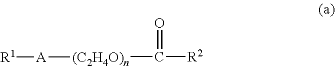
wherein in the formula (a), R¹ and R² each independently represent an alkyl group having 4 to 15 carbon atoms, A represents COO or O, and n represents 30 to 300.

2. The solution for contact lens according to claim 1, wherein R¹ and R² each independently represent an alkyl group having 11 carbon atoms, A represents COO, and n represents 50 to 200.
